(19) United States
(12) Patent Application Publication
(10) Pub. No.: US 2007/0009596 A1
(43) Pub. Date: Jan. 11, 2007

(54) Controlled drug release composition resistant to in vivo mechanic stress

(76) Inventors: Stefano De Luigi Bruschi, Milano (IT); Sergio Pengo, Bovisio Masciago (IT)

Correspondence Address: Mark P Levy;Thompson Hine, 2000 Courthouse Plaza NE, 10 West Second Street, Dayton, OH, 45402-1758, US

(21) Appl. No.: 10/556,734

(22) PCT Filed: May 14, 2004
(86) PCT No.: PCT/EP04/50814

§ 371 Date: Nov. 14, 2005

(30) Foreign Application Priority Data
May 14, 2003 (IE) 2003/0366

(51) Int. Cl. A61K 9/22

(52) U.S. Cl. 424/468

(57) ABSTRACT

The controlled release units of the present invention are capable of maintaining the drug/s sustained release properties along the gastro-intestinal tract without losing their mechanical resistance induced by the in vivo peristalsis. The formulations object of the present invention are based on a matrix consisting in a glyceryl ester in combination with ethers of cellulose. Such a matrix composition further incorporates the drug/s. The matrix units of the present invention may be obtained by known granulation methods or by direct compression according to the rheological properties of the drug/s. The drug release profile in vitro matches very well with release in vivo, i.e. the controlled release matrix is not damaged by in vivo peristalsis. The present compositions show good drug release properties, even at very early stages after administration, and ensure a constant and complete release of drug within acceptable times.

### FIELD OF THE INVENTION

The present invention relates to a pharmaceutical composition for drug controlled release characterised by an in vivo high mechanical stress resistance and an in vivo broad and regular time absorption profile.

### STATE OF THE ART

Swellable matrices are widely used to get monolithic or multiparticulate dosage forms capable of ensuring drug release profile according to the therapeutic needs. A mixture is made dispersing the drug with soluble or insoluble hydrophilic polymers plus compression adjuvants. The mixture is then granulated or directly tabletted to get the final controlled release dosage form. Drug release occurs thanks to the swelling properties of the polymer constituting the matrix that hydrates in presence of aqueous media thus exerting the drug release control.

According to the drug solubility, release mechanism is based on diffusion through the swollen matrix or by polymer erosion or a combination thereof.

Drug release kinetic is governed by several factors i.e. drug solubility, polymer hydration rate, polymer viscosity and loading, type and amount of fillers, etc.

An exhaustive description of these controlled release systems can be found on the U.S. Pat. No. 4,259,314 patent and U.S. Pat. No. 4,680,323 patent.

The matrix systems described in those patents are specifically designed to ensure an in vitro drug dissolution rate to give rise to the expected drug peak plasma levels after the intake.

It is well known for those skilled in the art that the choice of the type and quantity of the dissolution medium as well the stirring conditions adopted will depend upon the drug solubility and the absorption window.

In some cases the dissolution method adopted can be used to assess an in vivo-in vitro correlation (IVIVC). As a result, knowing the pharmacokinetic of the drug, peak plasma levels can be predicted by the drug dissolution rate data by means of mathematical convolution methods.

Based on the assumption that drug release from a controlled release dosage form is the rate-limitng step in the absorption process, the absorption time profile resulting from the mathematical convolution may be considered to be indicative of an in vivo dissolution (D. Young et al “in vitro-in vivo correlations” advances in experimental medicine and biology, vol, 423 Plenum Press, © 1997 New York and London).

After the intake, the ability of the dosage form manufactured by hydrophilic matrix system to stand the in vivo peristalsis, thus maintaining its controlled release properties along the gastrointestinal tract, is therefore essential to ensure the peak plasma levels expected.

Unfortunately, the in vivo poor mechanical resistance conferred to the swollen state by known hydrophilic matrix systems may be the cause of a pharmacokinetic distribution failure even if the in vitro drug dissolution rate complies with the defined specifications.

In fact the methodology based on the determination of the drug dissolution rate as a predictive tool to ascertain the in vivo peak plasma levels denotes severe limits since the common dissolution apparatus do not stress mechanically the dosage form that maintains a well defined shape during the dissolution test: on the contrary, in vivo the absorption profile generally shows a dramatics peak plasma level, due to the mechanic smashing of the dosage form. These high drug concentration can lead to undesirable physiological effects.

To get the expected peak plasma levels it becomes of paramount importance to provide the gellable dosage form with suitable mechanical properties to resist the in vivo peristalsis without affecting the dissolution properties leading to the desired in vivo absorption rate.

It is known from EP 0441245 to include hardened oils (i.e. hardened beef tallow, hardened rapeseed oil) into dosage form in order to increase its mechanical resistance. However the inclusion of these results in a general delaying of the release rate, thus rendering the drug less bioavailable, especially in the early times after administration

All the known controlled release matrixes of the prior art are not completely satisfactory in achieving an in vivo mechanical resistance. Therefore the need is felt for improved controlled release dosage forms being mechanically resistant against in vivo peristalsis and affording a desired release rate in vivo; in particular the need is felt to achieve an improved mechanical resistance in vivo without incurring the drawback of very prolonged release times, thus maintaining a prompt onset of action soon after administration, and releasing the entire drug dose within acceptable times.

### SUMMARY

The present application relates to a controlled drug release matrix consisting in a glyceryl ester, a cellulose ether and one or more drugs in specific weight ratios. Among glyceryl esters, preferably glyceryl behenate is chosen.

The cellulose ether is preferably hydroxypropylmethylcellulose, methylcellulose, hydroxypropylcellulose, hydroxyethylcellulose, cellulose acetate, their derivatives or mixture thereof.

Preferably the cellulose ether is characterised by apparent viscosity varying in the range of 15 cP to 100.000 cP (2% w/v aqueous solution, 20° C.).

As well, the present invention relates to a controlled drug release pharmaceutical composition wherein said matrix is mixed with pharmaceutically acceptable excipients and is formulated in an orally administrable form characterised by a mechanical resistance at the swollen state bigger than the same composition without glyceryl ester. This mechanical resistance leads to a better prediction of the in vivo drug plasma concentration based on the in vitro release kinetic studies.

Orally administrable dosage forms can be obtained by processes known per se: e.g. a mixture of cellulose ether, glyceryl ester and one or more drugs can be directly compressed or granulated, than tabletted, etc.

Finally, the present invention relates to the pharmacological exploitations of the described composition.

### DESCRIPTION OF THE FIGURES

FIG. 1. Dissolution rate of ISO-5-MN by controlled release tablet (100 mg/tablet).

In vitro release amount in percent (%), (vertical axis) during time (hours, horizontal axis) of reference G9A623 tablet (-∘-) and I9A010 tablet (-Δ-) according to the present invention. Tests are made respecting the USP XXV dissolution apparatus 2,500 ml pH 1.2, 75 rpm, n=6 tablets.

FIG. 2. Plasma concentration (fed condition) of controlled release ISO-5-MN tablet (100 mg/tablet)

In vivo plasma concentration (ng/ml, vertical axis during time (hours, horizontal axis) of reference G9A623 controlled release tablet (-□-) and I9A010 controlled release tablet (-∘-) according to the present invention. AUC (area under the curve): G9A623=11384 ng/ml×hr.; I9A010=11451 ng/ml×hr

FIG. 3. Mechanical resistance comparison of ISO-5-MN tablet (100 mg/tablet)

Mechanical resistance comparison (Force, N, vertical axis) at the swollen state during probe displacement (Distance, horizontal axis, mm) of reference G9A623 controlled release tablet (-) and I9A010 controlled release tablet (- -) according to the present invention. G9A623 AUC (0-7 mm): 29.9 N mm; I9A010 AUC (0-7 mm): 209.4 N mm. Instrument: TA-XT2 Texture Analyser, Stable Micro System® (Load Cell: 25 kg; Swelling time: 6 hours: Liquid medium: pH 6.8 USP; n=3 tablets).

FIG. 4. Dissolution profile of Levodopa from Carbidopa/Levodopa tablets (50/200 mg/tablet)

In vitro release amount in percent (%), (vertical axis) during time (hours, horizontal axis) of reference P003C085 tablets (-⋄-) and P003C124 (•••Δ•••) tablets object of the present invention. Tests are carried out using USP XXV Flow through apparatus 4, 16.7 ml/min, gradient, 1 hour HCl 0.1N, 1 hour HCl 0.01 N, 4 hours BP buffer pH 4, tablets n°=6.

FIG. 5. Levodopa plasma concentrations (fasted, dose: 1 tablet) of Carbidopa/Levodopa 50/200 mg controlled release tablets.

In vivo plasma concentration (ng/ml, vertical axis) during time (hours, horizontal axis) of reference P003CO85 controlled release tablet (-∘-) and P003C124 controlled release tablet (-□-) according to the present invention,

FIG. 6. Mechanical resistance on Levodopa dissolution of Carbidopa/Levodopa 50/200 mg controlled release tablets.

In vitro Levodopa release from Carbidopa/Levodopa 50/200 mg controlled release tablets subjected to USP XXV disintegration apparatus, 800 ml BP buffer pH 4.0, tablet n°=3). Reference tablet lot is P003C085 (•••□•••), object of the present invention is tablet lot P003C124 (-⋄-).

### DETAILED DESCRIPTION OF THE INVENTION

According to the present invention it has been discovered that, in order to overcome the poor mechanical resistance showed at the swollen state by hydrophilic matrix dosage forms, the inclusion in the matrix of a glyceryl ester renders the controlled release system less susceptible to mechanical damages. As a result, during the gastrointestinal transit, the dosage form does not lose its mechanical structure thus ensuring an absorption profile governed solely by the dissolution kinetic ensured by the hydro-lipophyilic matrix system.

The mechanical resistance leads to a better overlapping of in vitro-in vivo drug release profile, thus enabling a reliable prediction of drug plasma concentration based on in vitro dissolution rate.

The matrix composition object of these invention is characterised by a great amount of glyceryl ester: despite the use of large amount of this lipophilic component, hydro-lipophilic matrixes object of the present invention lead not only to an in vivo improvement of mechanical resistance and to a delay of drug absorption profile, but also to a significant release of drug at early stages after administration, thus avoiding a prolonged lag phase before the drug's effects can be perceived by the patient.

Among the glyceryl ester, glyceryl behenate is preferred.

Non limiting examples of cellulose ethers are hydroxypropylmethylcellulose, methylcellulose, hydroxypropylcellulose, hydroxyethylcellulose, cellulose acetate, their derivatives or mixture thereof. These cellulose ethers are commercialised in a number of different grades with different apparent viscosity and degree of substitution. Among them, hydroxypropylmethylcellulose (Methocel® K, Methocel® E) and methylcellulose (Methocel® A ) are the polymers preferred. Their apparent viscosity can vary in the range of 15 cP to 100,000 cP (2% w/v aqueous solution, 20° C.).

An exhaustive description of the physico-chemical properties of these polymers can be found on the Handbook of Pharmaceutical Excipients, third edition, edited by A. H. Kibbe, © 2000 American Pharmaceutical Association and Pharmaceutical Press.

In particular, it has been surprisingly found that a matrix made of drug, glyceryl ester and cellulose ether in the weight ratios described below, is capable to associate a very high mechanical resistance to a very quick onset of action immediately after the administration, thereby associating two mutually opposing principles. Even more surprisingly, the initial release rate was found to be even higher than the one produced by a comparative composition free of glyceryl ester. In these matrixes the drug accounts for about 20-95%, the glyceryl ester for about 1-25%, the cellulose ether for about 1-65% by weight of the matrix.

In a first preferred group of matrixes the drug accounts for about 20-70%, the glyceryl ester for about 1-25%, the cellulose ether for about 1-65% by weight of the matrix. In a more preferred subgroup the drug accounts for about 20-30% (preferred 25%), the glyceryl ester for about 15-25% (preferred 20%), the cellulose ether for about 45-65% (preferred 55%) by weight of the matrix.

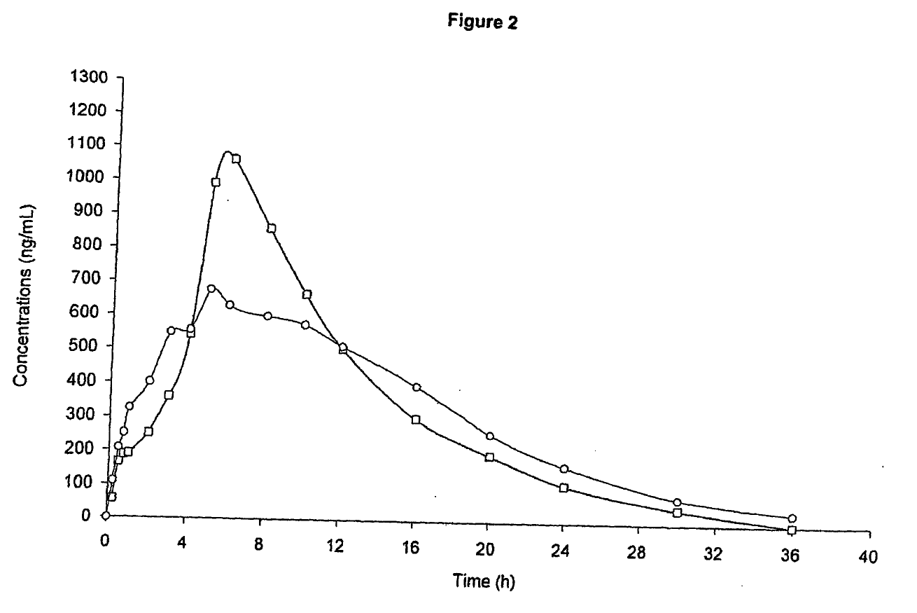

In a further preferred group of matrixes the drug accounts for about 70-95%, the glyceryl ester for about 1-15%, the cellulose ether for about 1-15% by weight of the matrix. Such matrixes are characterised by a sustained drug release profile that leads to a higher plasma concentration during hours far from administration, FIG. 2. The matrix is also characterised by releasing significant amounts of drug immediately after administration: therefore the mechanical hardening of the tablet did not result in delaying the start of the release process; moreover, quite advantageously, this early-stage drug release did not grow into any undesired release peaks.

According to another embodiment, the present invention provides a controlled release matrix containing carbidopa and/or levodopa as a drug, characterised by an ideal drug sustained release and by an exact reproducibility of in vitro data when used in vivo. In this embodiment the glyceryl ester and the cellulose ether are mixed with carbidopa and/or levodopa; said carbidopa and/or levodopa represent about 70-95%, preferably 80-95% by weight of the matrix; said glyceryl ester represents about 1-15% by weight of the matrix; said cellulose ether represents about 1-15% of the matrix; the matrix is further characterised by weight ratios of glyceryl ester to cellulose ether ranging from about 4:1 to 1:1.

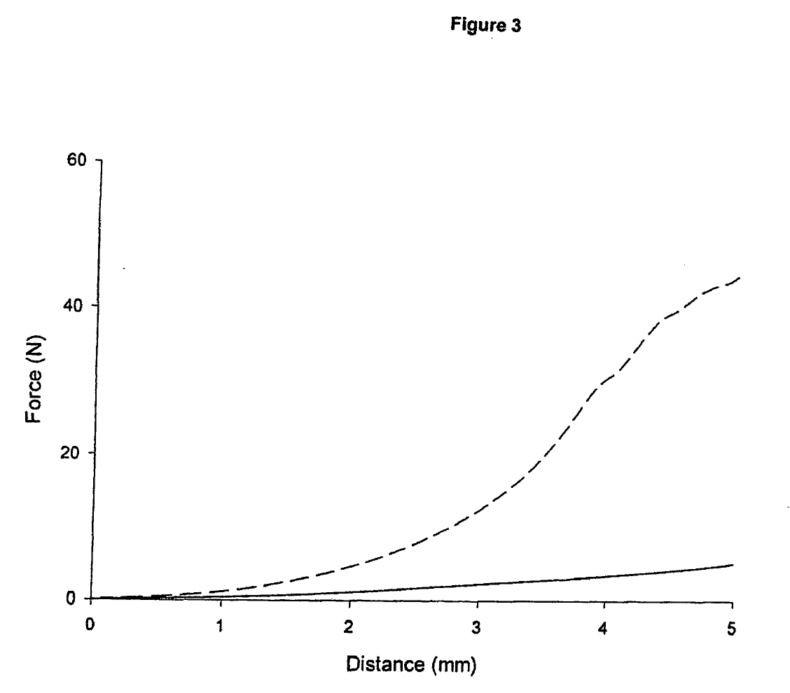

All the matrixes according to the present invention, suitably mixed with pharmaceutically acceptable excipients, can be formulated as oral dosage forms. Excipients include pH-buffering agents, polymeric excipients, i.e. carboxyvinylpolymers, tabletting adjuvants, binders, lubricants, colouring agent etc. These dosage forms are characterised by an improved mechanical resistance, as shown on FIG. 3. Among the method used to estimate the mechanical resistance of controlled release matrices at the swollen state are dynamometric measurements and dissolution kinetic measurement upon stress condition.

Dynamometric tests were made by a texture analyser model TA-XT2 equipped with a 9 mm diameter probe able measure the force that the swollen tablets opposes when pressed.

Dissolution kinetic measurements on stressed conditions were made by USP XXV disintegration apparatus. Tablets were placed in a basket rack immersed in a simulated gastrointestinal fluid.

Upon the apparatus was operated, liquid aliquots were withdrawn at specific time-points and analysed to assess the drug release.

The dosage forms are prepared using pharmaceutical processes namely by direct compression or by granulation processes and final tableting. The process comprises the steps of dispersing one or more drugs with one or more glyceryl esters and one or more cellulose ethers.

Suitably, pharmaceutically excipients are also added; the final mixture is than directly compressed or alternatively granulated before being compressed.

In detail, the production stepsare essentially:
mixing one or more drugs in appropriate amount with one or more glyceryl ester, one or more cellulose ethers, and possible tabletting adjuvants;
direct compression or granulation of the mixture;
tabletting.

Alternatively drugs can be granulated with a suitable binder, then granules are admixed with one or more glyceryl ester, one or more cellulose ethers and possible tabletting adjuvants: the final mixture is then tabletted.

In another embodiment of the present invention drug/s can be sprayed onto a mixture of the aforementioned components before tabletting, for instance according to the following sequence of steps:
mixing an appropriate amount of a glyceryl ester with a cellulose ether and possible tabletting adjuvants;
spraying an appropriate amount of one or more drugs and possible binder onto the mixture aforementioned;
compression of the mixture.

The hydro-lipophilic matrix object of the present invention applies to monolithic dosage forms such as tablets or multiparticulate dosage forms units such e.g. minitablets, filled into gelatine capsules.

The present invention applies to any acceptable pharmaceutical drug deliverable with controlled release systems.

The invention is further illustrated with the following non limitative examples.

Experimental Section

### EXAMPLE 1

Two lots of controlled release Isosorbide-5-Mononitrate (5-ISMN) 100 mg tablets were prepared.

Lot G9A623 was made by hydrophilic matrix, lot I9A010 was made by hydro-lipophilic matrix object of the present invention.

Their quali-quantitative compositions are illustrated in Table I.

TABLE I

| | | |
|---|---|---|
| | Lot G9A623 | Lot I9A010 |
| | Quantity | Quantity |
| Ingredients | (mg/tablet) | (mg/tablet) |

| | | | |
|---|---|---|---|
| 1. | 5-ISMN | 100.0 | 100.0 |
| 2. | Hydroxypropylmethyl- | 320.0 | — |
| | cellulose 4000 cP | | |
| 3. | Hydroxypropylmethyl- | — | 230.0 |
| | cellulose 100.000 cP | | |
| 4. | Carboxyvinylpolymer | 50.0 | 25.0 |
| 5. | Glyceryl Behenate | — | 80.0 |
| 6. | Polyvinylpyrrolidone K30 | 80.0 | 80.0 |
| 7. | Dicalcium phosphate | 60.0 | 115.0 |
| | anhydrous | | |
| 8. | Silicon dioxide | 5.0 | 5.0 |
| 9. | Mg Stearate | 10.0 | — |

Tablets, identical in shape and size and showing an average hardness of about 200 N, were obtained by direct compression and than subjected to a pharmacokinetic study after single administration with human volunteers (fed condition).

Release mechanism is based on drug diffusion through the swollen polymers and progressive erosion of the matrix.

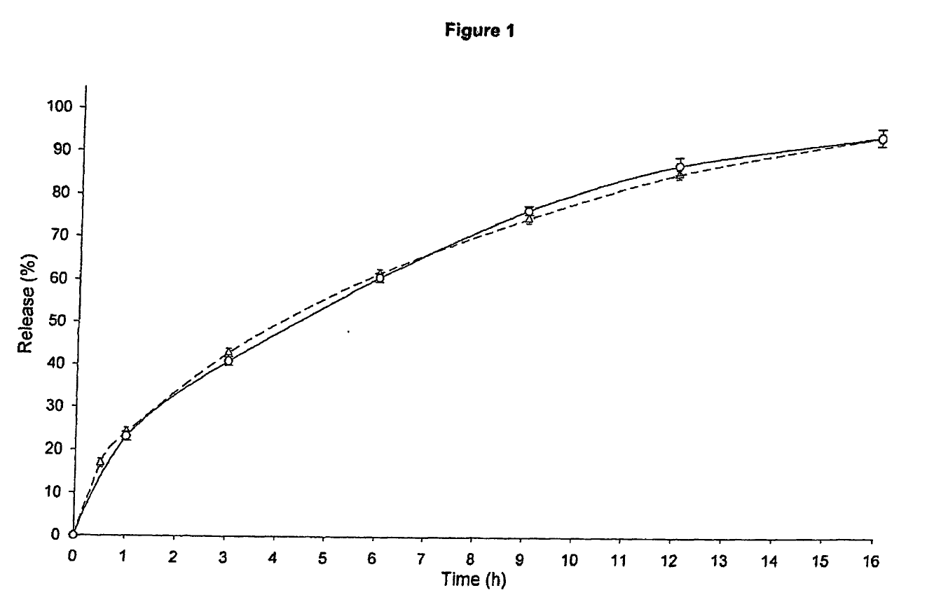

Tablet's in vitro drug dissolving rate and in vivo peak plasma levels are shown in FIGS. 1 and 2. From the graphs therein illustrated, it is evident that despite the in vitro drug dissolving rates of lots G9A623 and I9A010 were superimposable (FIG. 1), the in vivo peak plasma levels denoted a different absorption kinetic (FIG. 2). The huge peak plasma level evident in control G9A623 tablets during 0 to 12 hours is absent in I9A010 tablets, object of the present invention: in I9A010 tablets the plasma concentration profile during first times is greater than the control but the maximum concentration is about 30% lower. Besides, after 12 hours the plasma concentration due to I9A010 tablets is higher than G9A623 tablets.

The sharp plasma peak of G9A623 associated to the immediate decline of the plasma drug concentration is not peculiar to I9A010, which, ensuring a constant plasma drug concentration lasting about 6-8 hours, denotes a more regular time absorption profile. Surprisingly despite the more waxy structure of the tablet I9A010, the drug availability is greater from 0 hours to 4 hours in I9A010 tablet than in the G9A623 control tablet: this result shows that the composition object of the present invention doesn't slow the drug solubility rate, on the contrary it renders the drug available earlier.

Moreover, this features lead to a better prediction of the in vivo plasma drug concentration, based on the in vitro kinetic release: both the profiles of the I9A010 tablet are better overlapped the tablet's control profiles.

Besides different drug dissolving kinetics, very close areas under the curve (11384 ng/ml×hr Vs 11451 ng/ml×hr) indicate a similar effectiveness of ensuring the same extent of drug absorption for both the formulations.

Dynamometric tests on the two tablets were performed with a TA-XT2 texture analyser.

Tablets were allowed to swell for 6 hours at pH 6.8, than pressed at 1 mm/sec by a 9 mm diameter probe.

The force needed to penetrate the probe into the swollen tablet is a function of the distance (previously set in 7 mm according to the tablets height) and of its matrix texture.

A rapid force increment indicates the change from softer (hydrates) to harder material (ungelled-dry core), whereas the area under the curve values express the work done to ensure the probe penetration into the swollen tablets and can be considered as an indicator of the tablet's consistency.

The experimental results are represented in FIG. 3. Plots are indicative of a limited mechanical resistance at the swollen state by controlled release tablets G9A623 manufactured by a hydrophilic matrix in comparison with controlled release tablets I9A010 manufactured by a hydro-lipophilic matrix according to the present invention.

On the basis of the experimental data it is clear that at parity of drug dissolving rate, the mechanical resistance of controlled release systems at the swollen or hydrated state plays an important role on drug release.

Concerning the pharmacokinetic of the controlled release tablets lot G9A623 (FIG. 1), it becomes now clear that the sharp incline of the drug concentration starting from the fourth hour from the intake is due to a severe alteration of the drug release control of the swollen tablets induced by the in vivo peristalsis.

On the contrary, controlled release tablet lot I9A010 made by the hydro-lipophilic matrix object of the present invention due to their higher mechanical resistance at the swollen state ensures, at parity of drug dissolving rate, a constant and quick plasma level of the active agent for a prolonged time.

Especially surprisingly is the greater drug availability during the first 4 hours from the administration showed by the controlled release tablet lot I9A010 made by the hydro-lipophilic matrix object of the present invention, rather than the drug availability of the control tablet G9A623.

### EXAMPLE 2

Two lots of Carbidopa/Levodopa 50/200mg CR tablets were prepared.

Lot P003C085 was made by an hydrophilic matrix, lot P000C124 was made by a hydro-lipophilic matrix object of the present invention.

Their quali-quantitative compositions are illustrated in Table II.

TABLE II

| | | Lot P003C085 | Lot P003C124 |
|---|---|---|---|
| | | Quantity | Quantity |
| Ingredients | | (mg/tablet) | (mg/tablet) |
| 1. | Carbidopa monohydrate | 54.0 | 54.0 |
| 2. | Levodopa | 200.0 | 200.0 |
| 3. | Polyethylenglycol 6000 | 23.0 | 23.0 |
| 4. | Carboxyvinylpolymer | 0.6 | — |
| 5. | Polyvinylpyrrolidone K30 | 10.0 | — |
| 6. | Methylcellulose 15 Cp | — | 14.0 |
| 7. | Hydroxypropylmethyl- | — | 1.0 |
| | cellulose 4000 Cp | | |
| 8. | Glyceryl Behenate | — | 24.0 |
| 9. | Silicon dioxide | 2.2 | 3.0 |
| 10. | Mg Stearate | 2.2 | 3.0 |

Ingredients 1. and 2. were granulated with 3. Granules were then admixed with the remaining components and the mixtures compressed to get CR tablets having an average hardness of about 100 N. Tablets were subjected to pharmaco-kinetic studies with human volunteers (fasted condition). Because of the pH dependent solubility of the actives, a gradient dissolution analysis was made by the USP XXV flow through apparatus 4. Release mechanism is based on drug diffusion through the swollen polymers and progressive erosion of the matrix.

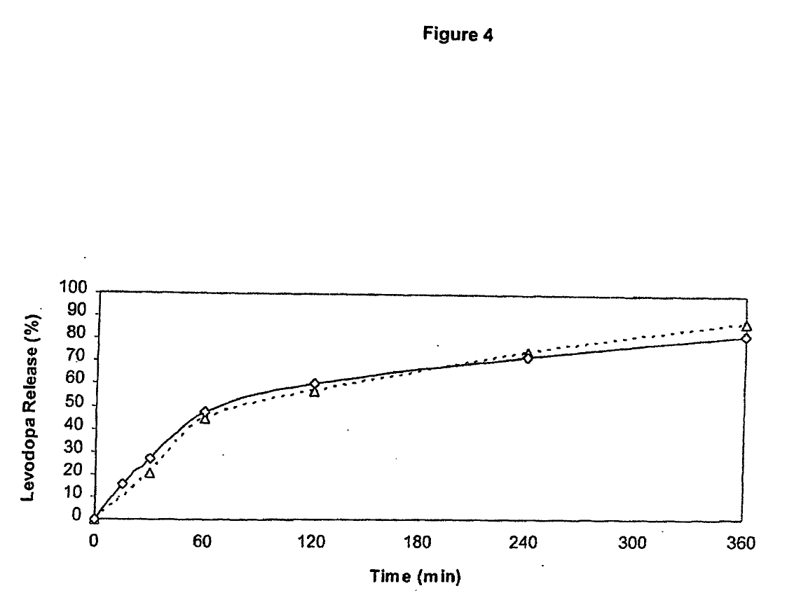

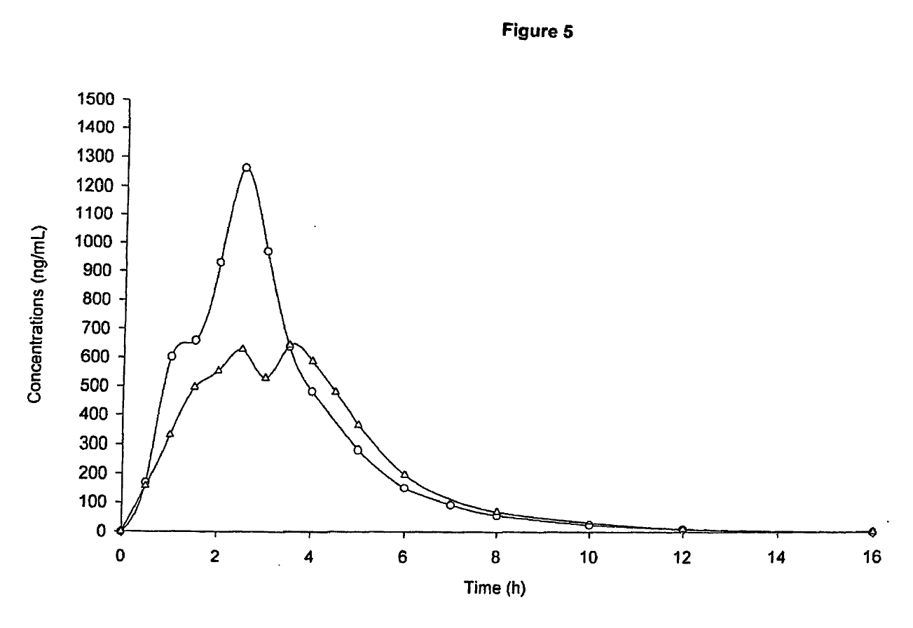

Levodopa in vitro release and peak plasma levels are shown in FIGS. 4 and 5. Similarly to what disclosed with Example 1, the hydrophilic matrix tablets lot P003C085 and the hydro-lipophilic matrix tablets lot P003C124 showed, at parity of in vitro dissolution profile (FIG. 4), a different in vivo time absorption kinetic (FIG. 5): the prompt and huge peak plasma level evident in hydrophilic tablets lot is absent in hydro-lipophilic tablets lot object of the present invention; on the contrary, in P003C124 tablets the drug plasma concentration after 4 hours is higher than in P003C085 control tablets, thus ensuring a more regular profile, maintaining constant plasma levels for a prolonged time.

To assess mechanical resistance of the two tablets lots, dissolution kinetic measurements on stress conditions were made by the USP XXV disintegration apparatus. A direct comparison was made between the hydrophilic matrix tablets lot P003C085 and the hydro-lipophilic matrix tablets lot P003C124 object of the present invention.

Tablets were placed in a basket rack immersed in a simulated gastrointestinal fluid buffered at pH 4 (BP). Upon the apparatus was operated, liquid aliquots were withdrawn at specific time-points and analysed to assess the drug release.

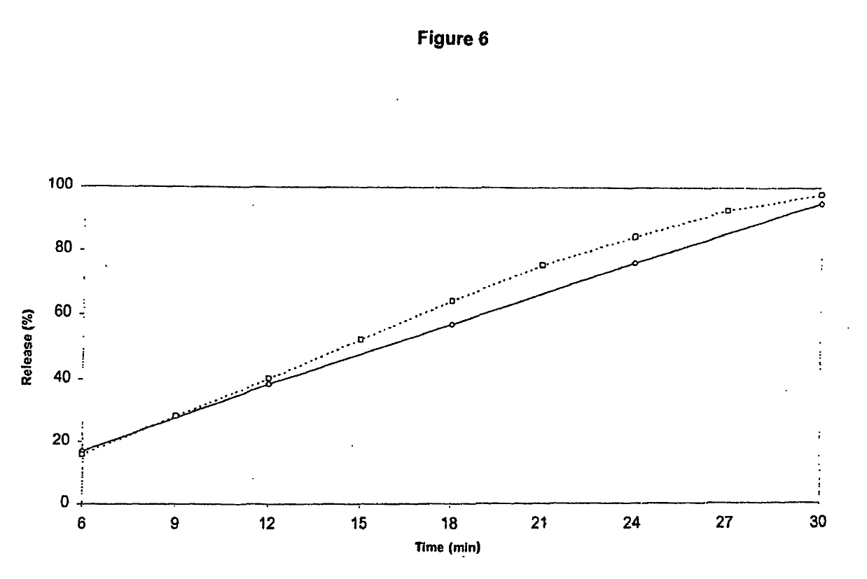

Results are shown in FIG. 6. The experimental data showed a faster dissolution kinetic for lot P003C085 (•••□•••) due to partial loss of its controlled release properties on stressed conditions. No significant mechanical alterations are observable for lot P003C124 (-⋄-) object of the present invention.

The higher mechanical resistance ensured by the hydro-lipophilic matrix composition of lot P003C124 did not affected the in vitro dissolution profile (FIG. 4) thus avoiding significant onset delay on drug absorption (FIG. 5).

### EXAMPLE 3

Two lots of controlled release ticlopidine hydrochloride tablets were prepared. Lot P004TH01 was made with the hydro-lipophilic matrix according to the present invention, lot P004TH02 was made with an hydrophilic matrix. Their quali-quantitative compositions are illustrated in Table III.

TABLE III

| | | Lot P004TH01 | Lot P004TH02 |
|---|---|---|---|
| | | Quantity | Quantity |
| Ingredients | | (mg/tablet) | (mg/tablet) |
| 1. | ticlopidine hydrochloride | 250 | 250 |
| 2. | glyceryl Behenate | 35.0 | — |
| 3. | hydroxypropylmethyl- | 75.0 | 75.0 |
| | cellulose 15.000 cP | | |
| 4. | microcrystalline cellulose | 45.0 | 65.0 |
| 5. | pregelatinized starch | 20.0 | 35.0 |
| 6. | povidone | 13.0 | 13.0 |
| 7. | anhydrous colloidal silica | 9.0 | 9.0 |
| 8. | Mg Stearate | 3.0 | 3.0 |

Tablets, having the same shape and size were obtained. The controlled release tablets manufactured with the hydro-lipophilic matrix according to the present invention (Lot P004TH01) showed higher mechanical resistance then the tablets manufactured with the hydrophilic matrix (lot P004TH02).

1. Controlled drug release matrix consisting in one or more drugs, a glyceryl behenate, and a cellulose ether.

2. Controlled drug release matrix according to claim 1, wherein the drug is present in amount of about 20-95%, the glyceryl behenate in amount of about 1-25%, and the cellulose ether in amount of about 1-65%, by weight of said matrix.

3. Controlled drug release matrix according to claim 2, wherein the drug is present in amount of about 20-70%, the glyceryl behenate in amount of about 1-25%, and the cellulose ether in amount of about 1-65%, by weight of said matrix.

4. Controlled drug release matrix according to claim 3, wherein the drug is present in amount of about 20-30%, the glyceryl behenate in amount of about 15-25%, and the cellulose ether in amount of about 45-65%, by weight of said matrix.

5. Controlled drug release matrix according to claim 4 wherein the drug amount is about 25%, the glyceryl behenate is about 20%, and the cellulose ether is about 55%, by weight of said matrix.

6. Controlled drug release matrix according to claim 2, wherein the drug is present in amount of about 70-95%, by weight of the matrix, the glyceryl behenate is present in amount of about 1-15%, by weight of the matrix, and the cellulose ether is present in amount of about 1-15%.

7. Controlled drug release matrix according to claim 6, wherein the drug is carbidopa and/or levodopa.

8. Controlled drug release matrix according to claim 7, wherein the weight ratio of glyceryl behenate to cellulose ether ranges from about 4:1 to 1:1.

9. Controlled drug release matrix according to claim 1, wherein the cellulose ether is selected from the group consisting of hydroxypropylmethylcellulose, methylcellulose, hydroxypropylcellulose, hydroxyethylcellulose, cellulose acetate, their derivatives and mixture thereof.

10. Controlled drug release matrix according to claim 1, wherein the cellulose ether is characterised by apparent viscosity varying in the range of 15 cP to 100.000 cP (2% w/v aqueous solution. 20° C.).

11. Controlled drug release pharmaceutical composition comprising the matrix claimed in claim 1, associated with pharmaceutically acceptable excipients and formulated in an orally administrable form.

12. Controlled drug release pharmaceutical composition according to claim 11, wherein the matrix represents at least 40% by weight of said pharmaceutical composition.

13. Controlled drug release pharmaceutical composition according to claim 11, wherein the matrix represents at least 60% by weight of said pharmaceutical composition.

14. Controlled drug release pharmaceutical composition according to claim 11, wherein excipients are selected from the group consisting of glidants, binders, flavours, preservatives, buffering agents, coloring agents, fillers, lubricants and combinations thereof.

15. Controlled drug release pharmaceutical composition according to claim 11, wherein the orally administrable form is a tablet, minitablet or granulate.

16. Process to prepare a controlled release composition, comprising the step of mixing together a drug, a glyceryl behenate and a cellulose ether.

17. Process according to claim 16, wherein the cellulose ether is selected from the group consisting of hydroxypropylmethylcellulose, methylcellulose, hydroxypropylcellulose, hydroxyethylcellulose, cellulose acetate, their derivatives and mixture thereof.

18. Process according to claim 16, wherein the cellulose ether is characterised by apparent viscosity varying in the range of 15 cP to 100.000 cP (2% w/v aqueous solution, 20° C.).

19. Process according to claim 16, wherein said drug, glyceryl behenate and cellulose ether are mixed with pharmaceutically acceptable excipients and the resulting final mixture is formulated into an orally administrable form.

20. Process according to claim 16, wherein the sum of said drug, glyceryl behenate and cellulose ether represents at least 40% by weight of said final mixture.

21. Process according to claim 16, wherein the sum of said drug, glyceryl behenate and cellulose ether represents at least 60% by weight of said final mixture.

22. Process according to claim 19, wherein excipients are selected from the group consisting of glidants, binders, flavours, preservatives, buffering agents, coloring agents, fillers, lubricants and combinations thereof.

23. Process according to claim 19, wherein the orally administrable form is a tablet, minitablet or granulate.

24. (canceled)

25. (canceled)
